(19) United States
(12) Patent Application Publication
(10) Pub. No.: US 2010/0069882 A1
(43) Pub. Date: Mar. 18, 2010

(54) MEDICAL DEVICE WITH PREFERENTIAL BENDING

(75) Inventors: Adam Jennings, Buffalo, MN (US); Ted Layman, Park City, UT (US); Tracee Eidenschink, Wayzata, MN (US); Matt Heidner, Maple Grove, MN (US); Clay Northrop, Salt Lake City, UT (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 12/233,078

(22) Filed: Sep. 18, 2008

(51) Int. Cl. A61M 25/00

(52) U.S. Cl. 604/525

(57) ABSTRACT

Medical devices such as catheters may provide advantages in flexibility, strength and other desired properties. Some medical devices may include a hypotube that has a plurality of slots disposed therein. The hypotube or another portion of the medical device may cause or permit the medical device to exhibit preferential bending in a single direction.

### TECHNICAL FIELD

The present invention relates generally to medical devices and more particularly to medical device that may be configured or which may include elements adapted to provide preferential bending.

### BACKGROUND

Medical devices such as catheters may be subject to a number of often conflicting performance requirements such as flexibility, strength, minimized exterior diameter, maximized interior diameter, and the like. In particular, often times there is a balance between a need for flexibility and a need for strength. Therefore, a need remains for improved medical devices such as catheters that are configured for an optimal balance between flexibility, strength, and other desired properties.

### SUMMARY

The present invention pertains to improved medical devices providing advantages in flexibility, strength and other desired properties.

Accordingly, an example embodiment of the present invention can be found in a medical device that includes a hypotube having a plurality of slots. The medical device may be configured to exhibit preferential bending in a single direction. While the medical device may not be excluded from bending in other directions, it should be understood that the medical device may preferentially bend in a single direction.

Another example embodiment of the present invention can be found in a medical device that includes a hypotube having a first side surface and an opposing second side surface. The first side surface includes a first plurality of slots disposed therein and the second side surface includes a second plurality of slots disposed therein. A restricting element is disposed along the first side surface.

Another example embodiment of the present invention can be found in a medical device that includes a hypotube having a first side and an opposing second side. A first plurality of slots are formed within the first side and a second plurality of slots are formed within the second side. The hypotube preferentially bends towards one of the first side and the second side.

Another example embodiment of the present invention can be found in a medical device that includes an elongate spiral cut member defining an exterior surface. A plurality of tethers are axially disposed about the exterior surface.

Another example embodiment of the present invention can be found in a medical device that includes a hypotube having a first side and an opposing second side. A first plurality of slots are formed within the first side and a second plurality of slots are formed within the second side. The first plurality of slots and the second plurality of slots are configured to cause the hypotube to preferentially bend towards one of the first side and the second side.

Another example embodiment of the present invention can be found in a medical device that includes a hypotube having a plurality of slots. Electroactive polymer segments can span at least some of the plurality of slots.

The above summary of the present invention is not intended to describe each disclosed embodiment or every implementation of the present invention. The Figures, Detailed Description and Examples which follow more particularly exemplify these embodiments.

### BRIEF DESCRIPTION OF THE FIGURES

The invention may be more completely understood in consideration of the following detailed description of various embodiments of the invention in connection with the accompanying drawings, in which:

FIG. 1 is a side elevation view of a catheter in accordance with an embodiment of the present invention;

FIG. 2 is a perspective view of a hypotube that may form a portion of the catheter of FIG. 1;

FIG. 3 is a perspective view of a hypotube that may form a portion of the catheter of FIG. 1;

FIG. 4 is a side elevation view of a hypotube that may form a portion of the catheter of FIG. 1;

FIG. 5 is a perspective view of a hypotube that may form a portion of the catheter of FIG. 1;

FIG. 6 is a side elevation view of a hypotube that may form a portion of the catheter of FIG. 1;

FIG. 7 is a perspective view of a hypotube that may form a portion of the catheter of FIG. 1;

FIG. 8 is a view of a spiral-cut hypotube that may form a portion of the catheter of FIG. 1; and

FIG. 9 is a cross-section taken along line 9-9 of FIG. 8.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “Labout”, whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following description should be read with reference to the drawings wherein like reference numerals indicate like elements throughout the several views. The drawings, which are not necessarily to scale, depict illustrative embodiments of the claimed invention.

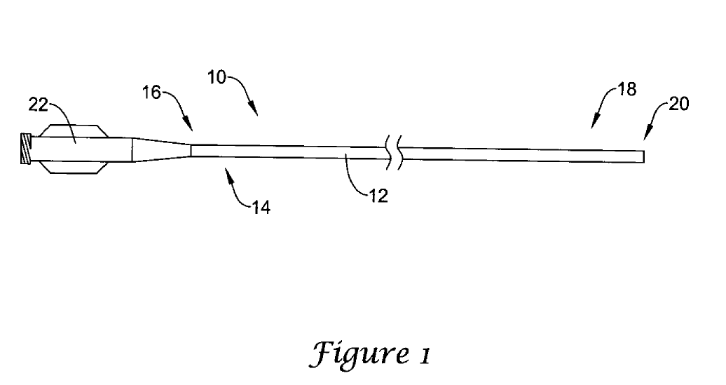

FIG. 1 is a plan view of a catheter 10 in accordance with an embodiment of the present invention. The catheter 10 can be any of a variety of different catheters. In some embodiments, the catheter 10 can be an intravascular catheter. Examples of intravascular catheters include balloon catheters, atherectomy catheters, drug delivery catheters, stent delivery catheters, diagnostic catheters and guide catheters. The intravascular catheter 10 can be sized in accordance with its intended use. The catheter 10 can have a length that is in the range of about 100 to 150 centimeters and can have any useful diameter. Except as described herein, the intravascular catheter 10 can be manufactured using conventional techniques.

In the illustrated embodiment, the intravascular catheter 10 includes an elongate shaft 12 that has a proximal region 14 defining a proximal end 16 and a distal region 18 defining a distal end 20. A hub and strain relief assembly 22 can be connected to the proximal end 16 of the elongate shaft 12. The hub and strain relief assembly 22 can be of conventional design and can be attached using conventional techniques. It is also recognized that alternative hub designs can be incorporated into embodiments of the present invention.

The elongate shaft 12 can include one or more shaft segments having varying degrees of flexibility. For example, the elongate shaft may include a relatively stiff proximal portion, a relatively flexible distal portion and an intermediate position disposed between the proximal and distal portions having a flexibility that is intermediate to both.

In some cases, the elongate shaft 12 may be formed of a single polymeric layer. In some instances, the elongate shaft 12 may include an inner liner such as an inner lubricious layer and an outer layer. If the elongate shaft 12 includes an inner liner, the inner liner can include or be formed from a coating of a material having a suitably low coefficient of friction. Examples of suitable materials include perfluoro polymers such as polytetrafluoroethylene (PTFE), better known as TEFLON®, high density polyethylene (HDPE), polyarylene oxides, polyvinylpyrolidones, polyvinylalcohols, hydroxy alkyl cellulosics, algins, saccharides, caprolactones, and the like, and mixtures and combinations thereof.

The elongate shaft 12 can include, as an outer layer or layers, any suitable polymer that will provide the desired strength, flexibility or other desired characteristics. Polymers with low durometer or hardness can provide increased flexibility, while polymers with high durometer or hardness can provide increased stiffness. In some embodiments, the polymer material used is a thermoplastic polymer material. Some examples of suitable materials include polyurethane, elastomeric polyamides, block polyamide/ethers (such as PEBAX®), silicones, and co-polymers. The outer polymer layer 32 can be a single polymer, multiple longitudinal sections or layers, or a blend of polymers. By employing careful selection of materials and processing techniques, thermoplastic, solvent soluble, and thermosetting variants of these materials can be employed to achieve the desired results. In some instances, a thermoplastic polymer such as a co-polyester thermoplastic elastomer, for example, available commercially under the ARNITEL® name, can be used.

In some cases, the catheter 10 may include reinforcing elements such as braids, micromachined hypotubes, and the like. In some instances, these reinforcing elements (not illustrated in FIG. 1) may be tailored to influence bending characteristics of the elongate shaft 12 or portions thereof. In some cases, it may be useful that the catheter 10 be adapted to preferentially bend within a single plane, or even in a single direction, for example.

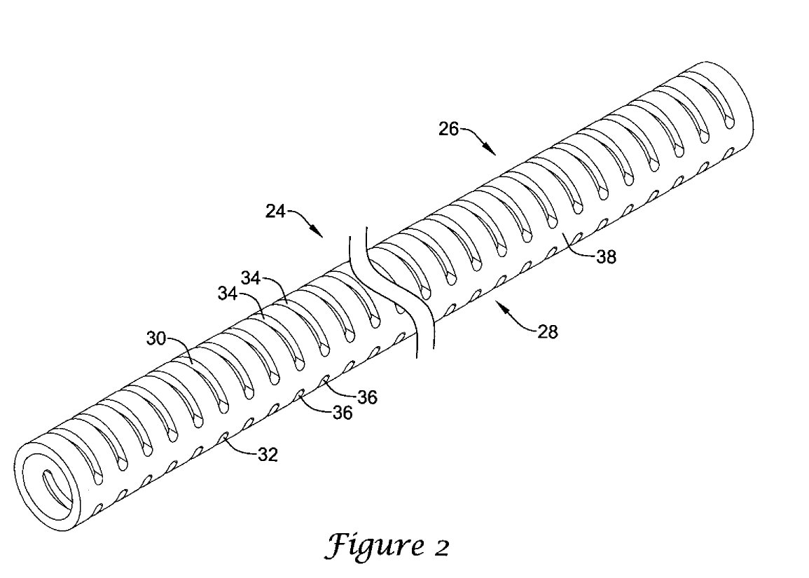

FIGS. 2 through 9 illustrate various reinforcing elements that, in accordance with particular embodiments of the invention, can instill the catheter 10 with preferential bending characteristics. The catheter 10 may be considered as including or being formed from the micromachined hypotubes, spiral-cut hypotubes and braids described hereinafter. In some cases, the catheter 10 may include one or more of these reinforcing elements within a distal portion of the elongate shaft 12. The reinforcing element(s) may be disposed within an interior of the elongate shaft 12, about an exterior of the elongate shaft 12, or between several layers forming the elongate shaft 12.

FIG. 2 is a perspective view of a micromachined hypotube 24 having a first side 26 and a second side 28. It will be appreciated that micromachined hypotube 24 is configured to preferentially bend within a single plane, i.e., towards the first side 26 (away from the second side 28) or towards the second side 28 (away from the first side 26). A first plurality of slots 30 are formed within the first side 26 of the micromachined hypotube 24. A second plurality of slots 32 are formed within the second side 28 of the micromachined hypotube 24.

In order to provide the micromachined hypotube 24 with single-planar bending characteristics, it can be seen that at least most of the individual slots 34 making up the first plurality of slots 30 are at least substantially radially aligned with the other individual slots 34. Similarly, at least most of the individual slots 36 making up the second plurality of slots 32 are at least substantially radially aligned with the other individual slots 36.

As a result of aligning the individual slots 34 and the individual slots 36, the micromachined hypotube 24 can be seen as having a first longitudinal rib 38 that extends axially along the micromachined hypotube. The first longitudinal rib 38 is formed or otherwise defined by the material remaining after the first plurality of slots 30 and the second plurality of slots 32 are cut into the micromachined hypotube 24.

A second longitudinal rib (not seen in this illustration) may be formed on an opposite side of the micromachined hypotube 24, radially spaced about 180 degrees from the first longitudinal rib 38. In some instances, the longitudinal ribs may be considered as defining a dividing plane between the first side 26 and the second side 28. It will be recognized that the micromachined hypotube 24 may preferentially bend within a plane that is perpendicular to a plane that extends axially along the micromachined hypotube 24 and through the first longitudinal rib 38 (and the second longitudinal rib, not shown).

In some instances, and as illustrated, an individual slot 34 or 36 may be rectangular in shape. In some instances, an individual slot 34 or 36 may be curved, such as a semi-circular shape. In some cases, an individual slot 34 or 36 may be diamond-shaped. An individual slot 34 or 36 may be formed using any suitable technique, such as saw cutting, a laser, or even by electrical discharge machining (EDM). Additional suitable techniques include chemical etching and abrasive grinding.

The micromachined hypotube 24 may be formed of any suitable polymeric or metallic material. In some cases, the micromachined hypotube 24 may be formed of a suitably stiff polymer such as carbon fibers, liquid crystal polymers, polyimide, and the like. In some instances, the micromachined hypotube 24 may be formed of a metallic material such as stainless steel or a nickel-titanium alloy such as Nitinol or other metallic or polymeric shape-memory material such as polycyclooctane. The micromachined hypotube 24 may include a combination of metal tubes and polymer tubes, if desired.

The micromachined hypotube 24 may be formed having any desired length, width, material thickness, and slot size as required to satisfy the requirements of any particular application. Additional details concerning the micromachined hypotube 24, including the manufacture thereof, can be found, for example, in U.S. Pat. No. 6,766,720 and published U.S. Patent Application No. 2004/0181174A2, each of which are fully incorporated, in their entirety, by reference herein.

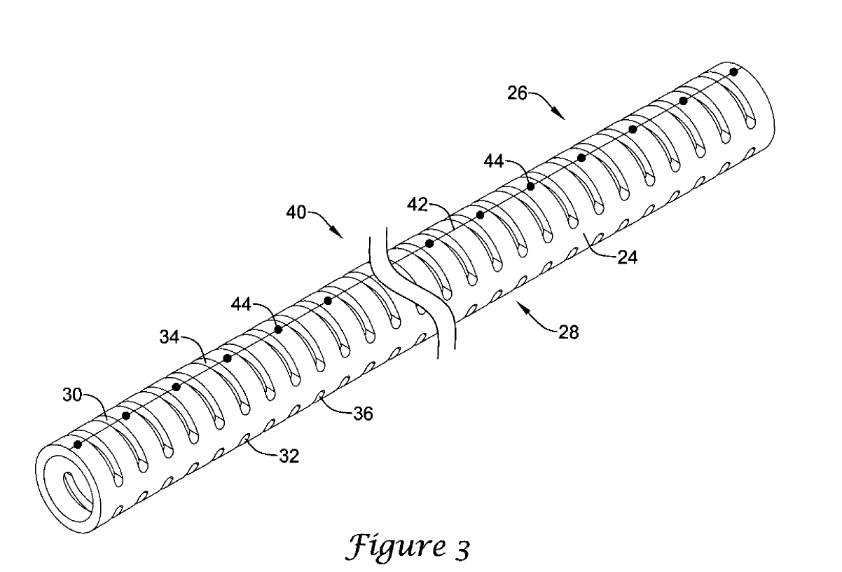

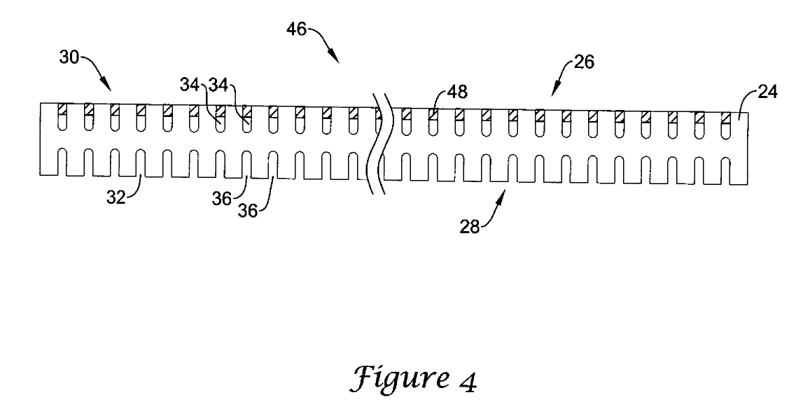

In some instances, it may be useful or beneficial to limit the micromachined hypotube 24 to preferentially bend in a single direction. FIGS. 3 and 4 provide illustrative but non-limiting examples of elements that may be added to the micromachined hypotube to provide preferential bending in a single direction.

FIG. 3 is a perspective view of an assembly 40 in which a restricting fiber 42 has been disposed along the first side 26 of the micromachined hypotube 24. The restricting fiber 42 is secured to the micromachined hypotube 24 via a plurality of attachment points 44. In some instances, the restricting fiber 42 is made of a relatively flexible but non-stretching material and thus permits the first plurality of slots 30 to close but not to open. As a result, the assembly 40 can only bend in a single direction (upward, in the illustrated configuration).

While the assembly 40 is shown with a single restricting fiber 42 extending for a substantial length of the micromachined hypotube 24, it will be appreciated that the assembly 40 may include several restricting fibers 42 axially disposed along differing portions of the micromachined hypotube 24 in order to provide the assembly 40 with desired bending characteristics.

In some cases, the restricting fiber 42 may be formed of a flexible but non-stretching metallic, polymeric or composite material. The restricting fiber 42 may be a single fiber, or a compilation of several smaller fibers or filaments. In some cases, the restricting fiber 42 may be a metallic strand. In some instances, the restricting fiber 42 may include or otherwise be formed of KEVLAR®.

The attachment points 44 may be formed in any suitable manner, using any suitable material. For example, if the restricting fiber 42 is a metallic strand or plurality of metallic filaments, the attachment points 44 may include welding attachments formed using any suitable technique such as laser welding. If the restricting fiber 42 is polymeric, the attachment points 44 may represent adhesive attachment points formed using any suitable adhesive. If both the restricting fiber 42 and the micromachined hypotube 24 are polymeric, the attachment points 44 may represent spots at which the reinforcing fiber 42 and the micromachined hypotube 24 are at least partially melted together.

FIG. 4 is a side view of an assembly 46 in which a polymeric element 48 has been added to the micromachined hypotube 24. In some instances, the polymeric element 48 includes a number of polymeric segments disposed within at least some of the first plurality of slots 30. In some cases, the polymeric element 48 may be formed by disposing a polymeric ribbon along the first side 26 and applying sufficient heat and/or pressure to soften or at least partially melt the polymeric ribbon into at least some of the first plurality of slots 30.

The polymeric element 48 may be formed of any suitable polymeric material. In some cases, the polymeric element 48 may be formed of a material that does not easily attach to the micromachined hypotube 24. In particular instances, the polymeric element may include or be formed of a polyethylene. When force is applied to the assembly 46, at least some of the first plurality of slots 30 will be able to open but not close. As a result, the assembly 46 may (in the illustrated configuration) bend downwards but not easily bend upwards.

While the assembly 46 is shown with the polymeric element 48 disposed in all or nearly all of the slots 34 within the first plurality of slots 30, it will be appreciated that the assembly 46 may include several distinct sections of the polymeric element 48 disposed along differing portions of the micromachined hypotube 24 in order to provide the assembly 46 with desired bending characteristics.

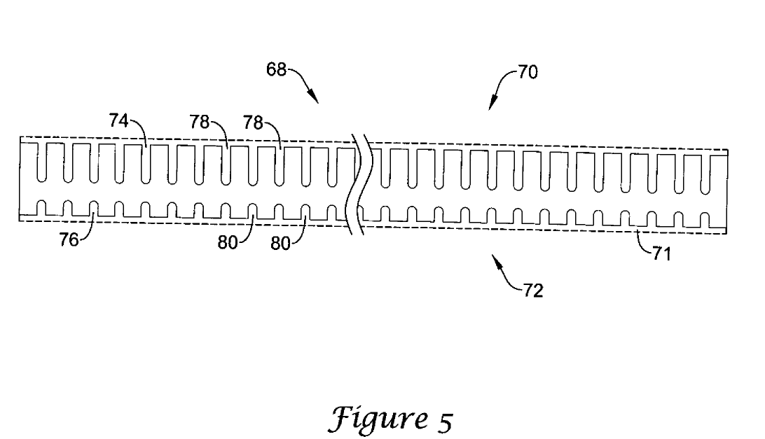

FIG. 5 is a perspective view of a micromachined hypotube 68 that can be considered as having a first side 70 and a second side 72. A first plurality of slots 74 are disposed along the first side 70 and a second plurality of slots 76 are disposed along the second side 72. The micromachined hypotube 68 may be formed of any suitable material and using any suitable technique as discussed with respect to the micromachined hypotube 24. A polymeric sheath 71 is disposed over the micromachined hypotube 68. The sheath 71 is shown in phantom to better illustrate underlying structure. The sheath 71 may be formed of any suitable polymer such as those discussed with respect to the elongate shaft 12 (FIG. 1).

It will be appreciated that the sheath 71 may enhance the preferential bending characteristics of the micromachined hypotube 68 since the sheath 71 would have a neutral bending axis while the micromachined hypotube 68 has a bending axis that is offset from an imaginary centerline. When the micromachined hypotube 68 bends, a portion of the sheath 71 will be in compression while another portion of the sheath 71 will be in tension. The cuts within the micromachined hypotube 68 that open when the micromachined hypotube 68 bends must open farther, causing greater strain in the sheath 71.

In some instances, as illustrated, at least some of the individual slots 78 making up the first plurality of slots 74 may be longer than at least some of the individual slots 80 making up the second plurality of slots 76. As a result, the micromachined hypotube 68 may be more likely to bend towards the second side 72 (downwards, as illustrated) and may be less likely to bend towards the first side 70 (upwards, as illustrated).

As illustrated, the first plurality of slots 74 and the second plurality of slots 76 extend across substantially all of the micromachined hypotube 68. In some cases, it is contemplated that the first plurality of slots 74 and/or the second plurality of slots 76 may extend only across a portion of the total length of the micromachined hypotube 68. The first plurality of slots 74 and/or the second plurality of slots 76 may extend discontinuously, i.e., in distinct segments, along the length of the micromachined hypotube 68.

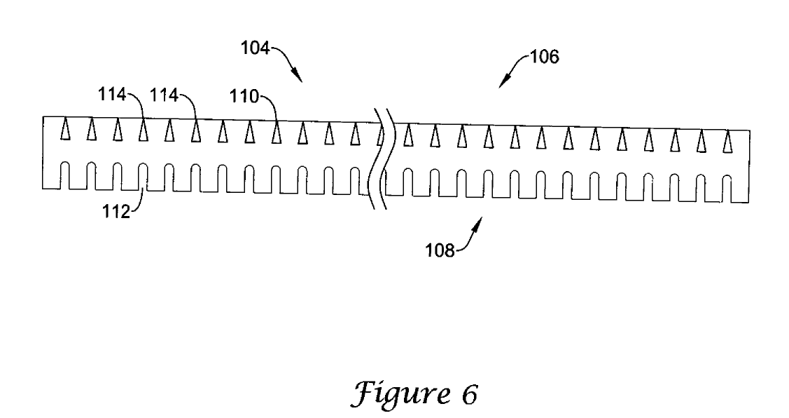

FIG. 6 provides an illustrative but non-limiting example of a micromachined hypotube 104 that may provide preferential bending in a single direction. The micromachined hypotube 104 has a first side 106 and a second side 108. A first plurality of apertures 110 are formed within the first side 106 and a second plurality of slots 112 are formed within the second side 108. The second plurality of slots 112 are formed similarly to those discussed with respect to the previous Figures. The micromachined hypotube 104 may be formed of any suitable metallic or polymeric material as discussed previously with respect to the micromachined hypotube 24.

The first plurality of apertures 110 may be formed having a configuration that allows at least some of the first plurality of apertures 110 to open but not to close. It will be appreciated, therefore, that the micromachined hypotube 104 will preferentially bend towards the second side 108 (downwards as illustrated) and will tend to not bend towards the first side 106 (upwards as illustrated).

In some instances, as illustrated, at least some of the individual slots 114 making up the first plurality of slots 110 may have a triangular shape. In particular, at least some of the individual slots 114 may have a width that is at a minimum at an outer surface of the first side 106 and that increases with relative closeness to a center of the micromachined hypotube 104.

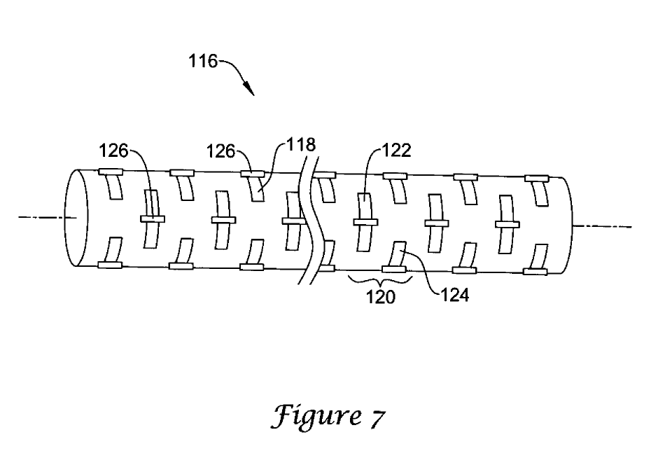

The micromachined hypotubes discussed herein have, for the most part, had a cutting pattern that preferentially limits bending to within a single plane. In some instances, a micromachined hypotube may have a cutting pattern that does not preferentially limit bending. FIG. 7 provides an illustrative but non-limiting example of a micromachined hypotube 116 that, by itself has no bending preferences.

The micromachined hypotube 116 has a plurality of slots 118 formed therein. It will be appreciated that the individual slots 118 may be considered as being in pairs 120, with a pair 120 including a first slot 122 and a second slot 124.

In some instances, as illustrated, the first slot 122 can have a first radial position on the micromachined hypotube 116 while the second slot 124 occupies a second radial position that is rotated from the first radial position. In some embodiments, as illustrated, the second slot 124 can be rotated about 90 degrees from the first slot 122. In other instances, the radial rotation can vary, especially if, for example, first slot 122 and first slot 124 are either longer or shorter than the illustrated length.

In order to control how the micromachined hypotube 116 bends or is otherwise shaped, the micromachined hypotube 116 can include one or more electroactive polymer segments 126 that can be disposed over at least some of the individual slots 118. The electroactive polymer segments 126 are adapted to change shape or size in response to an electrical stimuli. By either selectively locating the electroactive polymer segments 126, or by selectively activating only certain electroactive polymer segments 126, it can be seen that the shape of the micromachined hypotube 116 may be tailored for a specific use within a specific vascular anatomy.

An electroactive polymer is a polymer that, when subjected to a potential difference, accommodates ions which may cause the electroactive polymer to swell. In some cases, the electroactive polymer segments 126 may instead be formed of a shape memory material such as a shape memory metal or a shape memory polymer. Shape memory materials are known that can change from one configuration to another configuration upon a change in temperature, application of a magnetic field, light, or other suitable stimuli.

It should be recognized that since electroactive polymers accept or reject ions based on an applied potential difference, that the electroactive polymer segments 126 are reversibly altered between a position in which a specific electroactive polymer segment 126 has no impact on the shape of an individual slot 118, a position in which the specific electroactive polymer segment 126 has, for example, substantially closed the individual slot 118, and a plurality of intermediate positions.

For example, halting the potential difference being applied to the electroactive polymer will permit ions already within the polymer to remain there, but additional ions will not enter. Reversing the potential difference will cause the previously entered ions to exit the polymer. It should be recognized, therefore, that the relative amount of ions entering or exiting the electroactive polymer may be controlled by controlling the potential difference applied to the electroactive polymer.

As noted, in some instances, an electroactive polymer may be employed with hypotubes in accordance with certain embodiments of the invention. In short, an electroactive polymer is a doped polymer that undergoes volume or configuration changes upon oxidation and reduction, such as may occur when the polymer is subjected to an electrical field driving the ions into or out of the polymer. Oxidation and reduction may cause ions to be either inserted into the polymer, thereby increasing the volume of the polymer, or to be removed from the polymer, thereby decreasing its volume.

In some instances, an electroactive polymer may be doped with a large, immobile anion A− and may be positioned in contact with an electrolyte that includes a small mobile cation M+, in which case cations are inserted and de-inserted. The electroactive polymer, in this case, expands in volume in its reduced state (a negative potential). This can be represented as the following redox (oxidation-reduction) reaction:

P⁺(A⁻)+M⁺(aq)+e⁻ [symbol] P⁰(A⁻M⁺).

In some instances, the electroactive polymer can be polypyrrole that has been doped with dodecyl benzene sulfonate (DBS), and can be placed in contact with an aqueous electrolyte of 0.1 molar NaDBS (sodium dodecyl benzene sulfonate). In this case, DBS is the large, immobile anion and Na⁺, possibly hydrated, is the small cation that is inserted and or de-inserted into the polymer. During reduction, sodium cations move into the polypyrrole to achieve charge neutrality within the polypyrrole. On oxidation, conversely, the sodium cations are expelled from the polypyrrole.

Polypyrrole and NaDBS have the following chemical structures, respectively:

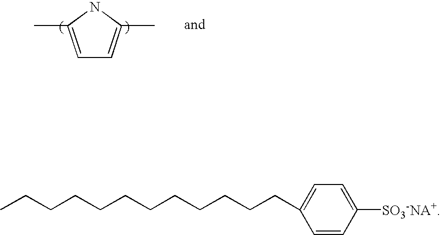

As noted, sodium cations can be provided by contacting the polypyrrole with an NaDBS electrolyte solution. However, in some instances, any variety of different aqueous salt solutions are useful. In particular, bodily fluids such as blood plasma and urine are effective.

In some cases, the electroactive polymer may be adapted to accommodate ions from a patient's own blood. In some cases, it may be useful to provide an electrolyte solution within an interior of the micromachined hypotube 116. Ions in general, and particularly cations, may flow (as a result of an appropriate potential difference) from either an electrolyte solution such as NaDBS or from a patient's blood into the electroactive polymer, thereby swelling or otherwise activating the electroactive polymer.

As noted, it is useful to provide a voltage or potential difference in order to drive the redox reaction discussed above. The oxidized state, in which the sodium cations have been expelled or at least largely expelled from the polypyrrole, can be achieved at a voltage of 0 volts, i.e. no applied current. The reduced state, in which the sodium cations have moved into the polypyrrole, can be achieved, for example, at a voltage of about 1 volts, or perhaps about 1.2 volts. It should be noted that intermediate voltages, say in the range of 0.4 to 0.6 volts, can cause an intermediate level of volume increase as a result of cations migrating into the polymer. Depending on the voltage applied, the polypyrrole may achieve a volume increase of at least about 30 percent.

Depending on how the electroactive polymer is employed, in some cases moving from the oxidized state to the reduced state, via application of an appropriate potential difference across the electroactive polymer, simply causes a volume increase, and the electroactive polymer merely swells or grows. In some cases, the electroactive polymer may be coupled with an electrode, such as in a gold/polypyrrole bilayer, and moving between oxidized and reduced states may cause the bilayer to either bend or straighten.

It will be recognized that in order to apply a potential difference (a voltage) to one or more of the electroactive polymer segments 126, two electrically conductive leads or conduits are needed. In some cases, particularly if the micromachined hypotube 116 is metallic, the micromachined hypotube 116 may itself serve as one of the electrically conductive leads. In some cases, an electrically conductive pattern (not illustrated) may be disposed on an interior or exterior surface of the micromachined hypotube 116. In some cases, a conductive wire may be disposed within an interior of the micromachined hypotube 116 to function as a second conductive lead.

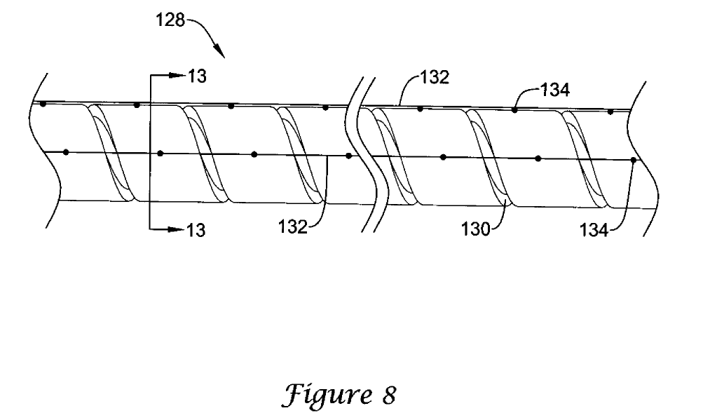

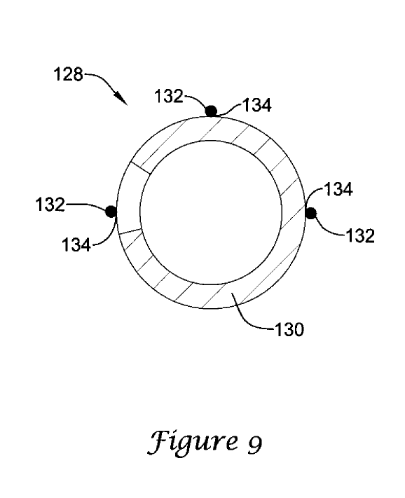

FIGS. 8-9 provide illustrative but non-limiting examples of structures, other than micromachined hypotubes, that can provide preferential bending to the catheter 10 (FIG. 1). FIG. 8 shows a side elevation of an assembly 130 while FIG. 9 provides a cross-section therethrough. The assembly 128 that includes a spiral-cut tube 130 and several tethers 132 that are secured to an exterior of the spiral-cut tube 130. In some cases, as best seen in FIG. 9, the assembly 128 may include a total of three tethers 132 that are positioned to influence the bending directions of the assembly 128. The spiral-cut tube 130 may be a metallic or polymeric tube that has been spiral-cut. In some cases, the spiral-cut tube 130 may instead be formed by coiling a flat ribbon, a round wire, or a filament having any other desired cross-sectional profile.

The tethers 132 may be formed of a flexible but non-stretching metallic, polymeric or composite material. Each of the tethers 132 may be a single fiber, or a compilation of several smaller fibers or filaments. In some cases, the tethers 132 may be a metallic strand. In some instances, the tethers 132 may include or otherwise be formed of KEVLAR®.

The tethers 132 are attached to the spiral-cut tube 128 at a plurality of attachment points 134. The attachment points 134 may be formed in any suitable manner, using any suitable material. In some instances, the attachment points 134 may include welding attachments formed using any suitable technique such as laser welding. In some cases, the attachment points 134 may represent adhesive attachment points formed using any suitable adhesive.

It will be appreciated that the tethers 132 may be positioned relative to each other to achieve a desired bending pattern. In the configuration shown in FIGS. 8 and 9, it can be seen that the assembly 128 will be permitted to bend upwards, but not downwards. As the spiral-cut tube 130 bends upwards, the tethers 132 will be permitted to collapse, thereby permitting the spiral-cut tube 130 to bend. However, as the tethers are at least substantially non-stretchable, the assembly 128 is not permitted to bend downwards (as illustrated).

In some embodiments, part or all of the devices described herein can include a lubricious coating. Lubricious coatings can improve steerability and improve lesion crossing capability. Examples of suitable lubricious polymers include hydrophilic polymers such as polyarylene oxides, polyvinylpyrolidones, polyvinylalcohols, hydroxy alkyl cellulosics, algins, saccharides, caprolactones, and the like, and mixtures and combinations thereof. Hydrophilic polymers can be blended among themselves or with formulated amounts of water insoluble compounds (including some polymers) to yield coatings with suitable lubricity, bonding, and solubility. In some embodiments, portions of the devices described herein can be coated with a hydrophilic polymer or a fluoropolymer such as polytetrafluoroethylene (PTFE), better known as TEFLON®.

The invention should not be considered limited to the particular examples described above, but rather should be understood to cover all aspects of the invention as set out in the attached claims. Various modifications, equivalent processes, as well as numerous structures to which the invention can be applicable will be readily apparent to those of skill in the art upon review of the instant specification.

We claim:

1. A medical device comprising:
a hypotube comprising a plurality of slots;
wherein the medical device is configured to exhibit preferential bending in a single direction.

2. The medical device of claim 1, further comprising an additional element that causes the medical device to exhibit preferential bending in a single direction.

3. The medical device of claim 2, wherein the additional element is secured to a side of the hypotube, and permits at least some of the slots on said side to open but not to close.

4. The medical device of claim 2, wherein the additional element is secured to a side of the hypotube, and permits at least some of the slots on said side to close but not to open.

5. The medical device of claim 1, wherein the hypotube itself is adapted to exhibit preferential bending in a single direction.

6. The medical device of claim 1, wherein at least some of the plurality of slots are configured to cause the hypotube to exhibit preferential bending in a single direction.

7. The medical device of claim 1, wherein at least some of the plurality of slots are sized to cause the hypotube to exhibit preferential bending in a single direction.

8. The medical device of claim 1, wherein at least some of the plurality of slots are shaped to cause the hypotube to exhibit preferential bending in a single direction.

9. A medical device comprising:
a hypotube having a first side surface and an opposing second side surface;
a first plurality of slots cut disposed within the first side surface;
a second plurality of slots disposed within the second side surface; and
a restricting element disposed along the first side surface.

10. The medical device of claim 9, wherein the restricting element comprises a polymeric ribbon melted into at least some of the first plurality of slots.

11. The medical device of claim 9, wherein the restricting element comprises a fiber secured to two or more locations along the first side surface.

12. A medical device comprising:
a hypotube having a first side and an opposing second side;
a first plurality of slots formed within the first side;
a second plurality of slots formed within the second side;
wherein the hypotube preferentially bends towards one of the first side and the second side.

13. The medical device of claim 12, wherein at least some of the first plurality of slots have a length that is equal to a length of at least some of the second plurality of slots.

14. The medical device of claim 12, wherein the hypotube has an interior surface and an exterior surface, and wherein at least some of the first plurality of slots have a triangular shape having a minimum width at the exterior surface.

15. A medical device comprising:
an elongate spiral cut member defining an exterior surface; and
a plurality of tethers axially disposed about the exterior surface.

16. The medical device of claim 15, wherein the plurality of tethers are not radially equidistantly disposed about the exterior surface.

17. The medical device of claim 15, comprising three tethers axially disposed about the exterior surface.

18. A medical device comprising:
a hypotube having a first side and an opposing second side;
a first plurality of slots formed within the first side;
a second plurality of slots formed within the second side;
wherein the first plurality of slots and the second plurality of slots are configured to cause the hypotube to preferentially bend towards one of the first side and the second side.

19. The medical device of claim 18, wherein at least some of the first plurality of slots have a length that is different from a length of at least some of the second plurality of slots.

20. The medical device of claim 18, wherein at least some of the first plurality of slots have a width that is different from a width of at least some of the second plurality of slots.

21. The medical device of claim 18, further comprising a polymer sleeve disposed about the hypotube.

22. A medical device comprising:
a hypotube comprising a plurality of slots; and
electroactive polymer segments spanning at least some of the plurality of slots.

23. The medical device of claim 22, further comprising a conductive pattern configured to selectively activate some of the electroactive polymer segments.
